(19) United States
(12) Patent Application Publication
(10) Pub. No.: US 2010/0312028 A1
(43) Pub. Date: Dec. 9, 2010

(54) MULTIPRODUCT BIOREFINERY FOR SYNTHESIS OF FUEL COMPONENTS AND CHEMICALS FROM LIGNOCELLULOSICS VIA LEVULINATE CONDENSATIONS

(76) Inventors: Edwin S. Olson, Grand Forks, ND (US); Carsten Heide, Grand Forks, ND (US)

Correspondence Address: MERCHANT & GOULD PC, P.O. BOX 2903, MINNEAPOLIS, MN, 55402-0903, US

(21) Appl. No.: 12/795,479

(22) Filed: Jun. 7, 2010

Related U.S. Application Data
(60) Provisional application No. 61/184,465, filed on Jun. 5, 2009.

(51) Int. Cl. C07C 1/22

(52) U.S. Cl. 585/242; 585/240; 585/310

(57) ABSTRACT

An integrated method for production of liquid transportation fuels, fuel additives, or chemicals in a biorefinery by the conversion of cellulosic materials is disclosed herein. The method is based on converting a source of C6 sugar such as cellulosic materials and sugars into a mixture of hydrotreated compounds. The biorefinery operates in a unique parallel-processing mode, wherein the initial biomass feedstocks are disassembled to provide substrates for parallel branches whose products may be reassembled in either a condensation step or a mixed hydrotreating step or a final fuel-blending step. The cellulosic materials can be converted to levulinate intermediates that condense with intermediates derived from other processes to produce fuels with the appropriate range of sizes in the target molecular composition, thus generating desirable combustion and physical properties. This method also makes use of methyltetrahydrofuran and other low carbon by-products that are separated for use as amphiphilic solvents. In an embodiment, the method produces cyclic ethers via mild hydrotreating of the condensation products, or long-chain keto ester, useful for plasticizers, by condensing a portion of the levulinate with a reagent containing an unsaturated group. In another embodiment, the method produces a ketal by converting a portion of the condensation product in an acid-catalyzed reaction with a diol.

### CROSS-REFERENCE TO RELATED APPLICATIONS

This application claims the benefit under 35 U.S.C. Section 119(e) of U.S. Provisional Application No. 61/184,456 entitled “Multiproduct Biorefinery for Synthesis of Fuel Components and Chemicals from Lignocellulosics via Levulinate Condensations,” filed Jun. 5, 2009, the disclosure of which is hereby incorporated herein by reference.

### STATEMENT REGARDING FEDERALLY SPONSORED RESEARCH OR DEVELOPMENT

This invention was made with government support under the Cooperative Agreement No. DE-FG36-08GO88054 entitled “EERC Center for Biomass Utilization 2009,” awarded by the U.S. Department of Energy. The government has certain rights in the invention.

### BACKGROUND

1. Field of the Invention

This invention is directed to an integrated process for production of liquid transportation fuels, fuel additives, or chemicals by the conversion of cellulosic materials. The fuels will be suitable for use in jet fuel, or diesel fuel; the fuel additives will be suitable for use in diesel fuel; the chemical will be suitable for use as plasticizers or amphiphilic solvents.

2. Background

More efficient means for conversions of agricultural, forest, aquaculture algae, and construction waste to fuels and chemicals are sought so that useful biomass-derived products can compete with and be integrated with the production of petroleum-based products. Although cellulose is the most abundant plant material resource, its exploitation has been curtailed by its composite nature and rigid structure. As a result, most technical approaches to convert lignocellulosic material to fuel products have focused on an effective pretreatment needed to liberate the cellulose from the lignin composite and break down its crystalline structure. Besides effective cellulose liberation, an ideal pretreatment has to minimize the formation of degradation products because of their wastefulness and inhibitory effects on subsequent processes. One way to improve the efficiency of biomass conversion schemes (biorefineries) is to integrate the energy-intensive lignocellulose depolymerization and dehydration (LDD) process with power production and/or other biomass processing. Many future biorefinery concepts rely on conversion of lignocellulose to glucose and subsequent fermentation, but this processing requires expensive enzymes and long contact times or produces inhibitors for the fermentation and low-value by-products. Fermentation releases carbon dioxide and produces cell mass, which may be usable only as a livestock supplement.

Alternative processing for lignocellulosic materials is acid-catalyzed depolymerization and conversion to the C5 product, levulinic acid, or levulinate ester. In general, two methods are used to produce levulinate from lignocellulose. One method uses water with a strong acid catalyst, such as sulfuric acid, to effect the depolymerization and dehydration of lignocellulose to produce the C5 and C1 acids (levulinic and formic acids) (see U.S. Pat. No. 5,608,105).

However, separation of products from the aqueous product solution is difficult. One patent describes a separation scheme that uses an olefin feed to convert the aqueous acid to esters that can be separated from the water and each other (see U.S. Pat. No. 7,153,996). Of course, a nearby olefin source is required for this process.

Another method uses an alcohol solvent for the acid-catalyzed depolymerization of cellulose, which results in direct formation of the levulinate ester (see DE 3621517).

A recent U.S. Department of Energy-sponsored project at the Energy & Environmental Research Center showed that high yields of methyl and ethyl levulinates along with charcoal and resins are obtained from several agricultural and wood (particleboard) wastes using relatively easy purification procedures, with little wastewater production. Valuable furfural and alkyl formates were also formed in addition to recovered resin from the particleboard and charcoal.

Several levulinic acid derivates have been proposed for fuel applications, such as ethyl levulinate, γ-valerolactone, and methyltetrahydrofuran. However, these components do not exhibit satisfactory properties when blended in petroleum-derived fuels.

Instead, valeric biofuels have been proposed by hydrogenation of γ-valerolactone to valeric acid, ethyl valerate, butyl valerate, and pentyl valerate (Angew. Chem. Int. Ed. 2010, 49, 1-6). The valeric platform potentially offers biofuels that can be used as components in both gasoline and diesel for blending. Nevertheless their acceptance as transportation fuels is challenged as they do not readily integrate in the existing petroleum fuel supply infrastructure.

The potential of levulinic acid and γ-valerolactone for biofuel manufacture has been also addressed by another method which converts γ-valerolactone into butenes via decarboxylation (see Science 2010, 327, 1110-1114). The butenes can provide a feedstock for gasoline but not for diesel or jet fuel unless they are further oligomerized. This multistep process seems to be too involved to be economically attractive.

Accordingly, a simple integrated method is needed to synthesize diesel and jet fuels, diesel additives, amphiphilic solvents, and plasticizers from C5 intermediates, levulinic acid, or levulinic esters with appropriate reagents that enable easy separation of product streams and simultaneously provide a mixture of the required hydrotreated higher molecular weight compounds.

### BRIEF SUMMARY OF SOME OF THE PREFERRED EMBODIMENTS

This invention comprises a set of integrated processes for achieving the desired goal of fuel and chemical production in a biorefinery. The biorefinery operates in a unique parallel processing mode wherein the initial biomass feedstocks are disassembled to provide substrates for parallel branches whose products may be reassembled in either a condensation step or a mixed hydrotreating step or a final fuel blending step as illustrated in various examples (FIGS. 1-6). In addition, the product streams of the biorefinery includes longer molecular weight products with a carbon chain length of 8 or higher created from the condensation step and shorter molecular weight by-products from unreacted starting materials.

Processing of the lignocellulosics can include their conversion to levulinate intermediates that condense with intermediates derived from other processes to produce fuels with the appropriate range of sizes in the target molecular composition, thus generating desirable combustion and physical properties.

One aspect of this invention is focused on the alternative catalytic processing of lignocellulose that directly produces good yields of a mixture of C5 and C1 esters or acids accompanied by valuable furfural and some carbon and resin. The catalytic processing of cellulosic biomass in alcohols offers a direct conversion to levulinate (C5) and formate (C1) esters that are useful for fuels and chemical intermediates. Levulinates are considered potential platform chemicals. The alkyl levulinates are valuable intermediates for formation of plasticizers.

Another aspect of this invention is the integration of a pyrolysis pretreatment step of cellulosic biomass. The biomass is depolymerized in such a thermal unit to give a soluble carbohydrate intermediate, such as anhydrosugars, prior to conversion to levulinate. In the thermocatalytic reaction, the anhydrosugars can be directly converted into ethyllevulinate or reagent aldehydes for the condensation step.

Another aspect of this invention is to convert the C5 acids or esters into fuel blendstocks for the production of finished fuels that meet petroleum-based fuel specifications. The present invention achieves this goal by integrating production of the levulinate derivatives with the processing of the disassembled noncellulosic portions of feedstock via a condensation of appropriate intermediates that results in a range of further intermediates with desired carbon chain lengths for fuels.

Another aspect of this invention is the integration of the reduction of fatty acid derivatives from the disassembled feedstocks with reduction of the condensation products to produce fuel blendstocks consisting of paraffins, isoparaffins, cycloparaffins, and alkylaromatics all of which are necessary for jet fuels to meet the physical fuel properties as specified for Jet-A or JetA1, for example.

Another aspect of this invention is production of cyclic ethers via mild hydrotreating of the condensation products. These cyclic ethers are utilized as diesel fuel additives to boost cetane value and reduce particulate emissions from the diesel combustion process. In some embodiments, this method is further integrated and uses the light cyclic ethers, such as methyl tetrahydrofuran, which occur as by-products, as solvent for the isolation of the levulinate products from the depolymerization reaction.

In some embodiments, this method integrates the catalytic processing of lignocellulosic materials. In order to meet the rigid specification for jet fuels, a fuel must comprise some of each of the types of hydrocarbons described above, as well as an appropriate distribution of carbon chain lengths. Blending of the streams from the parallel processing biorefinery accomplishes the final integration piece.

### BRIEF DESCRIPTION OF THE DRAWINGS

For a more detailed description of the preferred embodiment of the present invention, reference will now be made to the accompanying drawings, wherein:

FIG. 1 is a schematic of an integrated C5 biorefinery for oil seed biomass conversion to fuels via levulinate and isobutyraldehyde.

FIG. 2 is a schematic of an integrated C5 biorefinery for lignocellulose conversion to fuels via ethoxymethylurfural or furfural.

FIG. 3 is a schematic of an integrated C5 biorefinery employing the products and by-products for conversion to fuels.

FIG. 4 is a schematic of an integrated C5 biorefinery utilizing fruit and sugar beet wastes and a solid acid conversion unit for the soluble portion.

FIG. 5 is a schematic of an integrated C5 biorefinery for algae biomass conversion to fuels via ethyl levulinate and ethoxymethyl furfural or furfural.

FIG. 6 is a schematic of an integrated C5 biorefinery for lignocellulose conversion to fuels via anhydrosugars and levulinate.

FIG. 7 is a schematic of the depolymerization/decomposition of cellulose in ethanol and sulfuric acid, followed by a condensation reaction of ethyl levulinate with an aldehyde.

FIG. 8 is a schematic of a condensation product with furfural and subsequent Diels-Alder reaction and reduction to cycloparaffin.

FIG. 9 is a schematic of hydrogenation of levulinate intermediates:

A. Severe hydrogenation to alkanes,

B. Hydroisomerization to isoparaffins, and

C. Mild hydrogenation to alkyl tetrahydrofurans.

FIG. 10 is a schematic for the extraction and purification of the product mixture in unit (150) from reactor (100).

### DETAILED DESCRIPTION

### 1. Integrated Biorefinery

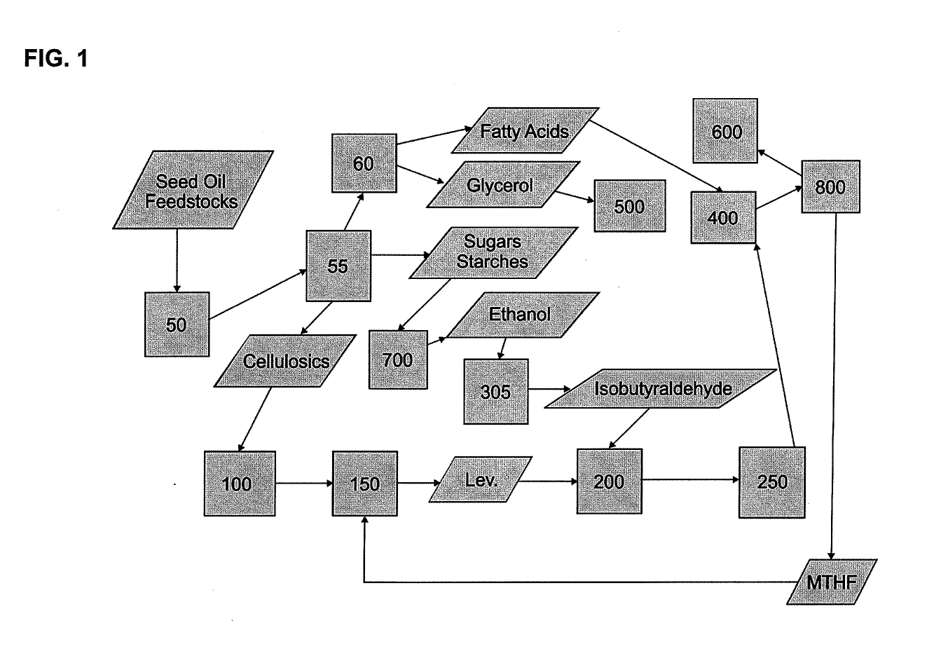

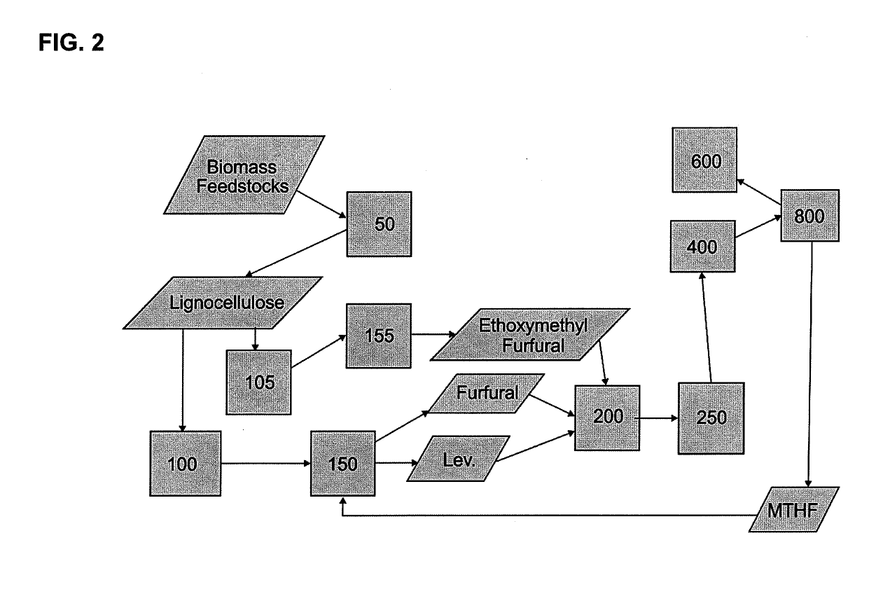

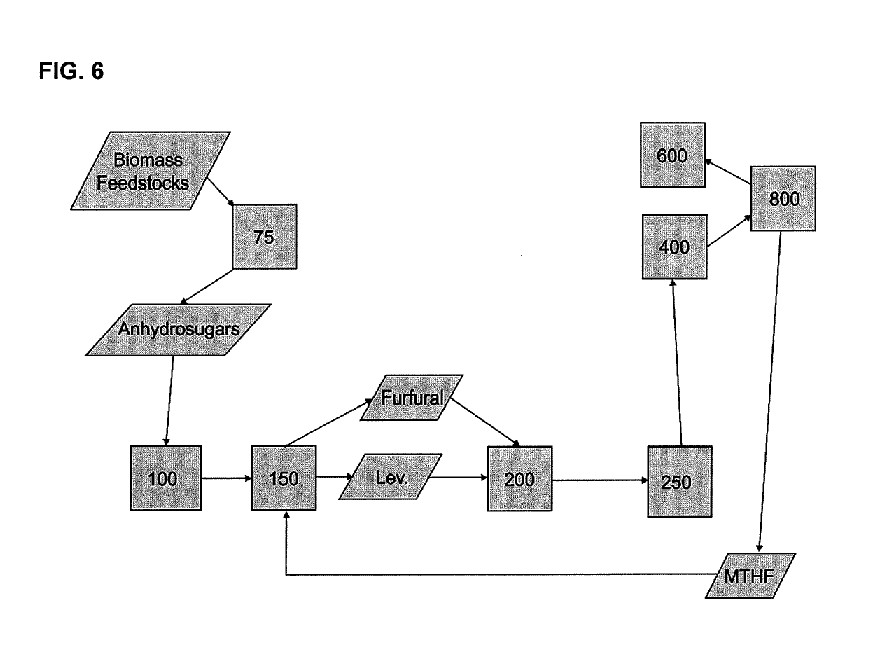

As illustrated in FIGS. 1 and 2, one of the preferred embodiments for the parallel processing C5 biorefinery is an integrated biorefinery comprising an initial separation (disassembly) unit (50 and or 55) for certain types of biomass containing oil where noncellulosic feedstocks are separated from cellulosic or lignocellulosic feedstocks, a cellulose depolymerization and dehydration (CDD) unit (100) that catalytically depolymerizes and decomposes or reforms the lignocellulose; a condensation unit (200) that condenses the primary product from the first unit with reactant aldehyde, ester, and ketone intermediates produced in a reagent production unit (300) from preferably renewable resources; and a hydrotreating unit (400) that converts the condensation products to fuels via hydrotreating. Additional units are added to convert by-products to chemical feedstocks and to separate and blend fuel components. Preferably, a separation unit (150) is added between the first (100) and second unit (200). Other energy crops, such as algae, are processed similarly (FIG. 6).

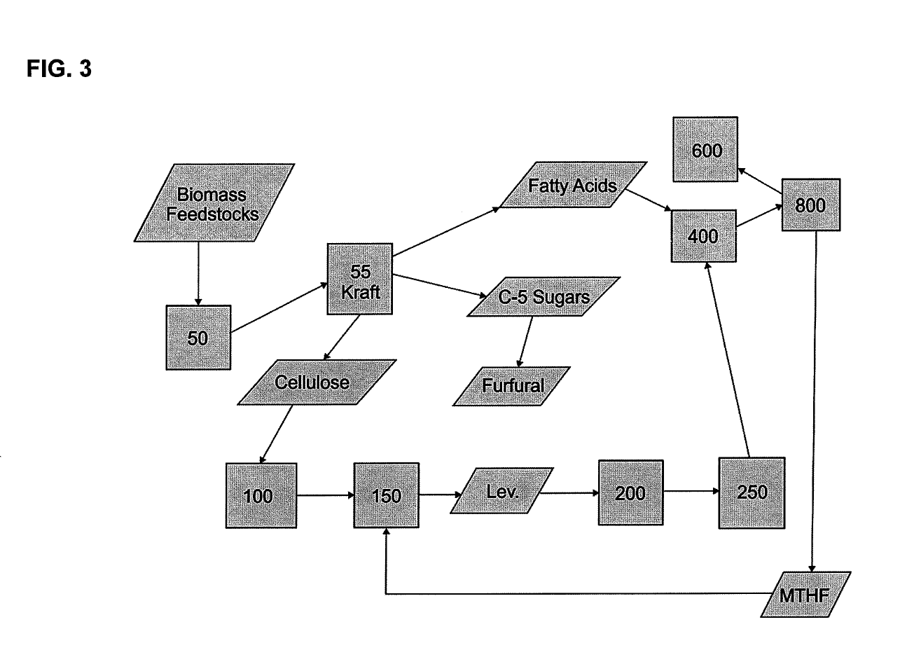

Alternatively, the process uses abundant cellulosic or lignocellulosic feedstocks (FIGS. 2, 3) comprising very low cost or negative cost wood and agriculture residue or grass and other energy crops. Lignocellulosic feedstocks are low in nitrogen and sulfur. The key to processing lignocellulosics to hydrocarbon fuels is the removal of the large amount of oxygen without carbonizing or polymerizing the carbon structures or expending a lot of hydrogen. The catalytic conversion to a levulinate (C5) intermediate is highly efficient in producing a material appropriate for further chemical synthesis because of the functionality retained in the first conversion.

Subsequent catalytic condensation reactions of the levulinate in the second unit (200) permit its conversion to higher molecular weight species (see FIGS. 1-8). Thus the 5-carbon acyl group of the ester is combined with aldehydes and ketones to form (5+x)-carbon products. The condensation reaction enables a simple separation of the (5+x) carbon products from the residue because of their lower solubility in water. Some of the levulinate condensation products will undergo a second cyclic condensation (Dieckmann condensation) to produce cyclic ketones. In order to prevent that the aldehydes, esters, and ketones undergo primarily a self-condensation reaction, it is important to choose x larger than 3. For providing suitable C9-C16 condensation products that are suitable for use in diesel and jet fuel after hydrotreating them, x should be in the range of 4 to 11.

Important for the integrated processing scheme are the syntheses of reagents for the condensation with the levulinate produced in a variety of ways from the separation products or by-products of the initial processing. In one embodiment (FIG. 1), ethanol from fermentation (700) of starches is converted to isobutyraldehyde (305) and used in the condensation reaction in the second unit (200).

In a further embodiment, the sugars and starches are used as a substrate for the production of hydroxymethylfurfural, alkoxymethylfurfural, and alkyl levulinates (FIG. 2). In these reactions, an aqueous or alcohol solution of the sugar or starch is pumped through a bed of solid acid catalyst.

The final integration occurs in the hydrogenation of the condensation products; the hydrotreating unit (400) gives both linear and branched hydrocarbons of appropriate chain lengths for JP-8 and other fuels. In addition, cycloparaffins are available from Dieckmann and Diels-Alder reactions of the intermediates prepared from ethyl levulinate. Low molecular weight cyclic ethers from hydrotreating are returned as solvent for the earlier separation.

### 2. Initial Separation (Disassembly) (50, 55, 60)

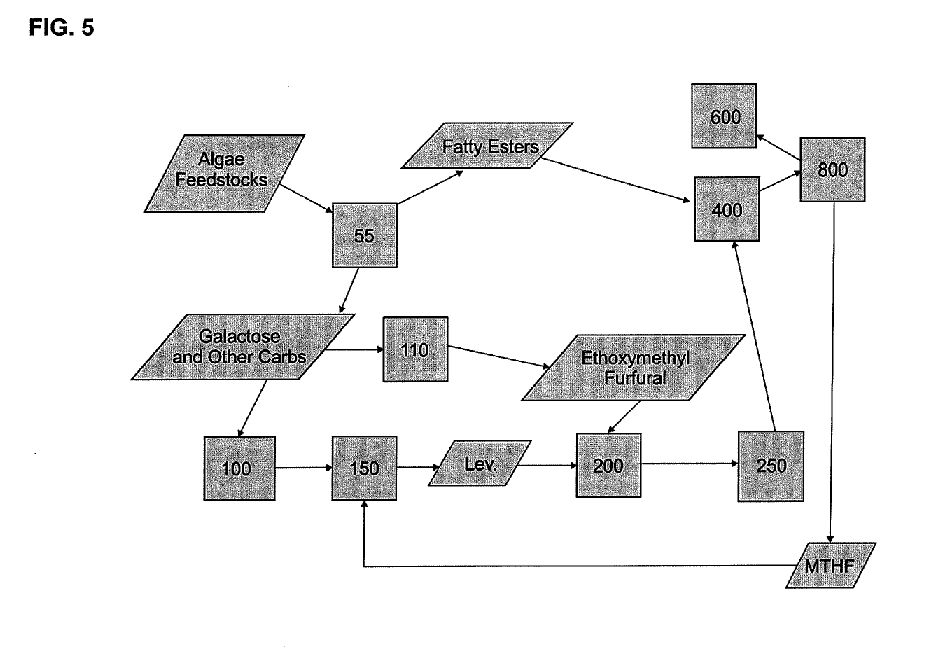

In an embodiment of this invention, where the feedstock is an oil seed such as corn, or the mechanical pretreatment unit (50) may be a wet mill which separates out the fibrous cellulosic material, from the starches and germ plasm, the germ plasm is treated by an oil extraction unit (55). The oil extraction unit (55) may be a press, more preferably a hexane- or CO₂-based extraction unit (see FIGS. 1 and 5). The starches and sugars may be fermented in fermentation unit (700) to produce alcohols, in particular, ethanol.

When the feedstock is algae, as illustrated in FIG. 5, the extraction is combined with transesterification to produce fatty acid esters: methyl (FAME) or ethyl (FAEE).

When integrated with a Kraft process, as illustrated in FIG. 3, the oil extraction unit (55) may yield tall oil fatty acids by first separating the raw tall oil soap from the spent black liquor by decanting the soap layer formed on top of the liquor storage tanks and then further extraction of the fatty acids. In an alternative embodiment, the tall oil soap is only filtered. The extracted oil, fatty acids, or tall oil soap may then be hydrotreated in the fourth unit (400).

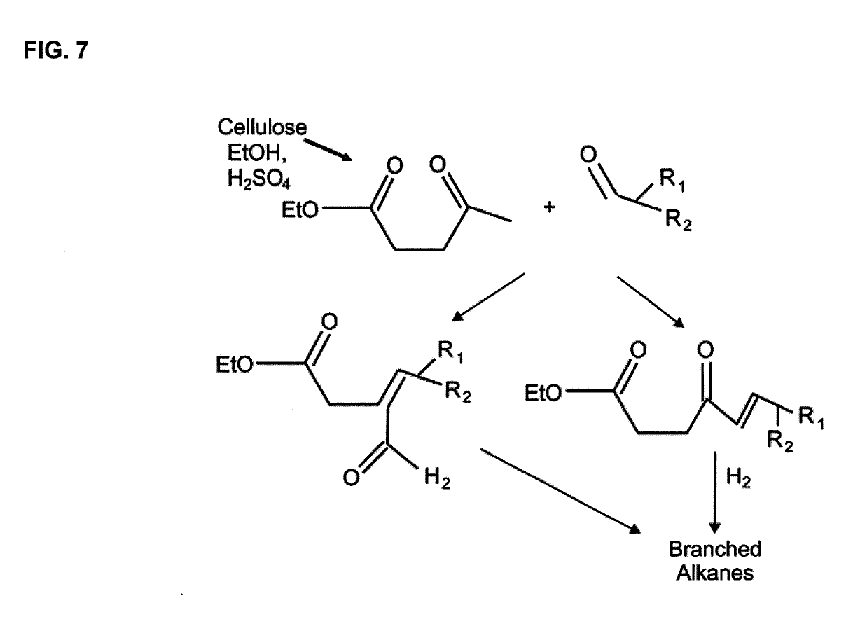

In another embodiment, the biomass feedstock comprises a cellulosic or lignocellulosic material, such as wood, wood pulp, pulping sludge, particleboard, paper, grasses, agricultural by-products such as straw, stalks, cobs, beet pulp, seed hulls, bagasse, or algae, any of which could be a by-product or waste form of the material (see FIGS. 3-6). These are reduced to a small, preferably granular size for the catalytic processing through a mechanical pretreatment unit (50). This pretreatment can be, for example, a simple mill or steam explosion gun. In another embodiment, the milled lignocellulose is further heated rapidly in a reactor (75, FIG. 7) to produce a condensable product comprising anhydrosugars, furfural, and lignin-based oils, which are separated.

### 3. Catalytic Depolymerization/Dehydration Unit (100)

Processing lignocellulosics to hydrocarbon fuels can include the removal of the large amount of oxygen without carbonizing or polymerizing the carbon structures or expending a lot of hydrogen. The present invention takes advantage of the acid-catalyzed mild thermal processing of levulinate units that maintain the type of oxygen functionality desired for further synthetic reactions.

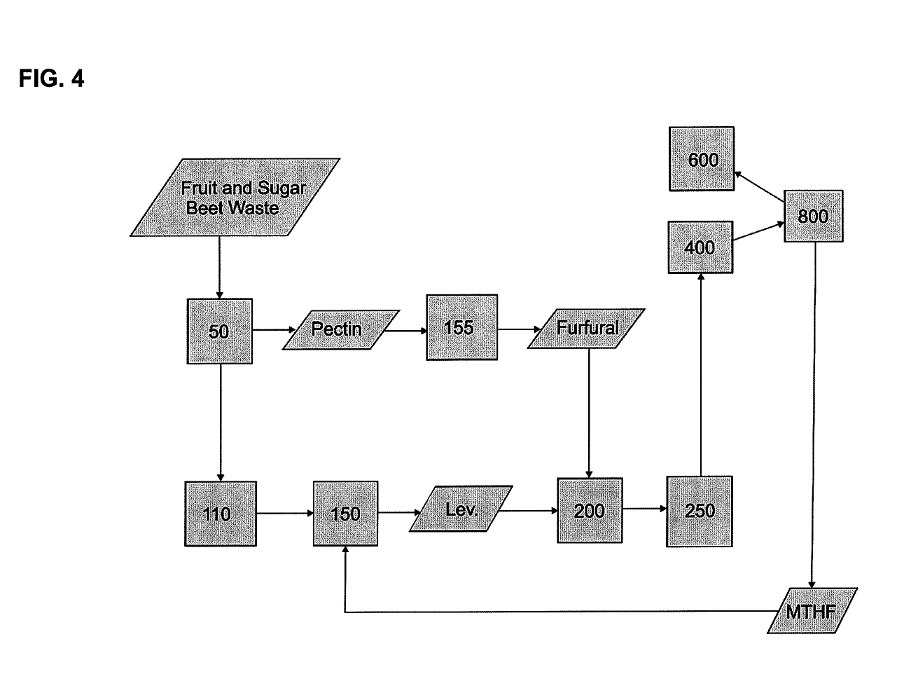

The catalytic depolymerization/dehydration unit utilizes a heated reactor (100) preferably at 120°-200° C. with a liquid or dissolved form of catalyst (preferably sulfuric acid) in FIGS. 1-3. A heated reactor with a solid acid catalyst bed is utilized in FIGS. 4, 5, and 6 where the feedstock is soluble or depolymerized and dehydration to the levulinate form is desired. Feedstock for producing levulinate may be any source of C6 sugar such as cellulosic materials and starches. Examples of sources of C6 sugars that may or may not be pretreated include wood, wood pulp, pulping sludge, particleboard, paper, grasses, agricultural by-products such as straw, stalks, cobs, beet, beet pulp, seed hulls, bagasse, algae, corn starch, potato waste, sugar cane, and fruit wastes, any of which could be a by-product or waste form of the material or a combination thereof.

Integration with a power plant or a recovery boiler can furnish low-pressure (waste) steam to generate the desired temperatures for the different reactors.

The reactor of the first unit (100) may be a pressurized autoclave or, preferably, a continuous reactor. The preferred embodiment in this invention is the continuous reactor, wherein a slurry of the biomass feedstock in acidic water or alcohol is pumped or augured through the heated reactor under mild pressure and wherein the residence time in the reactor is between 20 and 60 minutes.

The catalytic depolymerization/dehydration unit can be run with either of two different liquid streams: aqueous or alcoholic. In the aqueous medium, equal molar amounts of levulinic acid and formic acid are produced and are soluble in the aqueous acid. In case of lignocellulosic material processing, furfural is also formed from 5-carbon sugars present in the hemicellulose and is removed as overhead and collected during the processing. Separation of the acid products from the aqueous acid solution and from each other is difficult. However, for some acid-catalyzed processes, the process can continue through the next step without separation of the acids because separation is more easily effected on a more hydrophobic product from the subsequent reaction. Only the insoluble char and tars are separated, for example, with a filter and solid- or liquid-phase extraction, respectively. The furfural may be purified by distillation. Levulinic acid may be vacuum-distilled along with some of the water, or it may be extracted from the aqueous acid with an ether or ester solvent, such as methyltetrahydrofuran or gamma valerolactone, derived from the process in a later hydrogenation step. The insoluble char and tar may be further dewatered and may be thermally converted in a recovery boiler to provide process heat or fed to a power plant.

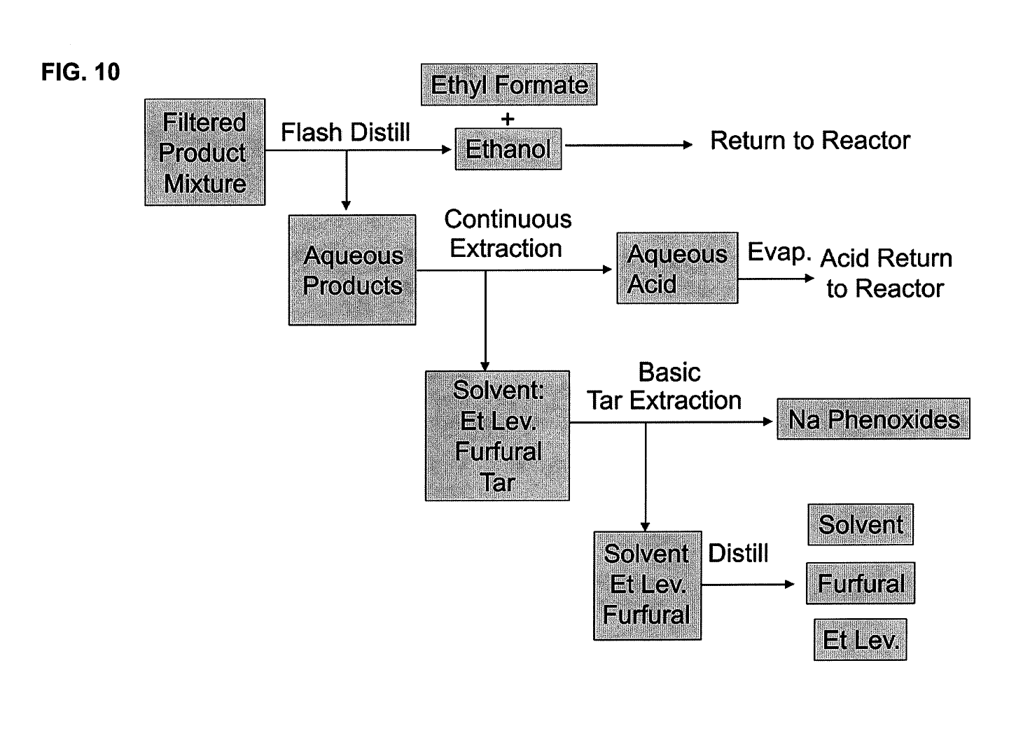

The reaction medium for the depolymerization/dehydration can also comprise an acid alcohol solution, such as that obtained by adding sulfuric acid and methanol or ethanol. Ethanol may come from the fermentation unit (700). The products of the reaction are methyl levulinate and methyl formate or the corresponding ethyl esters (FIG. 7). Longer-chain alcohols also can be used as the liquid medium, but they give lower yields of the ester products. The depolymerization/dehydration in ethanol of particleboard and other waste materials to ethyl levulinate ester proceeds in good yield when conducted in ethanol with sulfuric acid catalyst at 200° C. (FIGS. 1-7). Compared to the similar preparation of levulinic acid using an aqueous acid medium, the ethyl levulinate is more easily purified by (FIG. 10) extraction and/or distillation and can be easily separated from the concomitantly formed furfural (from the 5-carbon units present in the hemicellulose and ethyl formate). A preferred solvent for the extraction of levulinate esters and levulinic acid is methyltetrahydrofuran, produced in the hydrotreating unit (400) from ethyl levulinate or levulinic acid remaining in the condensation product mixture. Another preferred solvent is γ-valerolactone, which is also produced in the hydrotreating unit (400) from the same source.

Another embodiment for the first unit (100) is to distill the levulinic acid product so as to form angelica lactone (see FIG. 2). The angelica lactone is highly reactive in subsequent condensation reactions, owing to the acylation reactivity of the enolic lactone group, and also provides a route to products substituted at the alpha position.

In another embodiment for the first unit (100), the depolymerization/dehydration is conducted at a lower temperature, wherein ethoxy (or methoxy)methylfurfural is formed in addition to the levulinate. This intermediate is used directly in the condensation reactor or is converted to chemical products and monomers, such as furan dicarboxylate.

### 4. Condensation Unit (200)

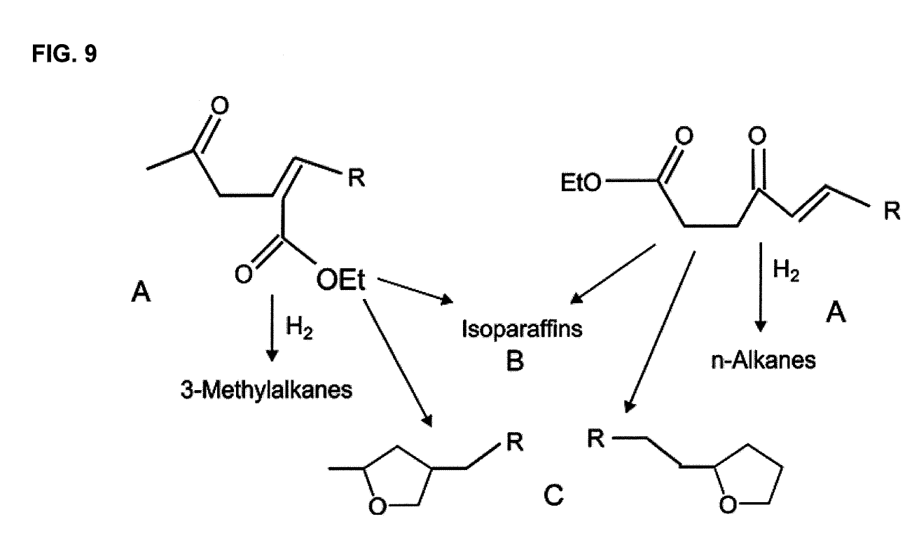

The third unit (200) in the integrated system is the reactor for conducting acid- or base-catalyzed condensation reactions (FIG. 7) of the C5 levulinate to produce higher molecular weight species with the chain lengths desired for jet fuel, diesel, amphiphilic solvents and plasticizers. Thus the 5-carbon acyl group of the levulinate is combined with aldehydes, esters, or ketones (Cₓ) to form (5+x)-carbon products. The condensation reaction is illustrated in FIG. 7. In FIG. 7, a branched aldehyde condenses with levulinate to form a mixture of branched ketoesters which are then hydrogenated to form branched alkanes or cyclic ethers. The latter reaction is shown in FIG. 9. In order to prevent that the aldehydes, esters, and ketones undergo primarily a self-condensation reaction, it is important to choose compounds with reactive carbonyl groups and unreactive alpha carbons that are branched or aromatic at this position. This implies that x is greater than 3. For providing suitable C9-C16 condensation products that are suitable for use in diesel and jet fuel after hydrotreating them, x should be in the range of 4 to 11. In addition, the aldehydes, esters, or ketones need to be branched to reduce the potential of any self-condensation. Also, for producing jet fuel, branched or aromatic aldehydes, esters, or ketones are preferred to produce a highly isoparaffinic fuel blendstock or cycloparaffinic fuel blendstock, respectively, that when blended together meet such important jet fuel criteria as freeze point, flash point, energy density, and physical density.

Another important aspect of this invention is that the fuel, solvent, and plasticizer must comprise an appropriate distribution of carbon chain lengths to provide for the proper distillation curve for the fuel, the amphiphillic character of the solvent, and the highly elastic features of a polymer from the use of the plasticizer, respectively. Therefore, the relevant aldehydes, esters, and ketones are derived from a limited group of feedstocks and chemical reactions that lead to the required carbon chain length distribution. Feedstocks for the reagent branched aldehydes are alcohols, such as isobutyl alcohol, that are produced by Guerbet reactions of ethanol and subsequently dehydrogenated to aldehydes, and olefins, for example from a petroleum refinery, that are converted to aldehydes by the oxo reaction. Aryl aldehydes are furfural, hydroxymethylfurfural, and substituted benzaldehydes that are produced from 5 and 6 carbon sugars or from lignin, respectively. Cyclic aliphatic aldehydes are produced by Diels-Alder reactions of acrolein (from dehydration of glycerol) with butadiene (from petroleum cracking or from ethanol via the Lebedev reaction). Reactive ketones include those with an adjacent carbonyl (1,2 diketones, 1,2 ketoesters) that are produced by fermentation or pyrolytic reactions of levulinic or, lactic acid. Vinyl esters are also highly reactive reagents; the one utilized in this invention is angelica lactone produced by distillation of levulinic acid over a mineral acid.

The condensation reaction of leuvelinats have precedence in the chemical literature, but these isolated reaction were not recognized for the potential for fuel or fuel additives synthesis. These reactions include the following: Benzaldehyde and substituted benzaldehydes (Erdman, Kato, Sen, Borshe), furfural (Ludvig & Kehler, Sen; Erdmann), isobutyraldehyde (Meingast), and self-condensation (Zotchik, Blessing), formaldehyde (Olsen) and phenol (Mauz). A recent patent application teaches the dimerization of levulinic acid on a cation exchange resin to form C10 units (Blessing, WO 2006/056591). The reaction proceeds in very low yields, 15% as reported. An older publication reports essentially the same process with a simple sodium base (Zotchik). This application instead utilizes an integrated process where levulinate esters are condensed with aldehydes in high yields and the condensation products are converted to cyclic ether diesel additives and hydrocarbons.

Product formation and separation are facilitated at this stage because of the low solubility of the longer-chain reaction products in water. Thus when levulinic acid from the first-stage aqueous reaction containing the acid catalyst is reacted with the aldehyde mixture, the products from the second unit (200) are now more easily extracted from the water with the solvent methyltetrahydrofuran. The acidic aqueous layer contains formic acid in addition to the sulfuric acid. Formic acid is vacuum-distilled along with some of the water in the separation unit (250), and the sulfuric acid catalyst is then recycled to the first dissociation/depolymerizaton unit after partial evaporation of the water content. Thus the integration of these two steps allows convenient product separation as well as a means of recycling the acid catalyst. No neutralization is needed. Aldol condensation products from the reaction of levulinic acid and an aldehyde conducted with an acid catalyst typically are a mixture of the β-(or branched) and the δ-(or unbranched) forms, as shown in FIG. 7. To achieve more of the δ-(or unbranched) form, a basic catalyst must be used. This is not feasible without removing the sulfuric acid used in the first-stage unit. Thus an alternative route is used for synthesis of unbranched isomers with an alkaline catalyst. Although some of the aldehyde undergoes self-aldol condensation, the products from this side reaction do not need to be removed since they are also converted to usable fuels in the final step.

The alternative synthesis route uses an alcohol such as methanol or ethanol in the first-stage depolymerization/dehydration unit (100) along with the soluble acid catalyst. Following the formation of the esters in the first-stage unit (100), the esters are extracted and separated by simple distillation—formate ester boiling at low temperature—alcohol and solvent are removed, then furfural. The higher boiling levulinate ester could be distilled or reacted without purification.

The levulinate ester that is formed in the alternative depolymerization/dehydration unit when alcohol is the vehicle for the biomass slurry is reacted with the aldehyde intermediates using a strong base catalyst to produce mainly the longer-chain esters. Preferably the catalyst for the condensation is a solid base catalyst so that a continuous reaction over the bed of the catalyst is performed, and no catalyst separation or neutralization is needed. The catalyst is preferably a hydrotalcite or a hydrotalcite impregnated with a basic material, such as potassium fluoride. When a soluble catalyst is employed, the catalyst must be removed from the product solution. Typically, the condensate product comprises a mixture of isomeric forms. For example, isobutyraldehyde is attacked by enolate carbanions formed at the delta and beta positions of the levulinate. The proportion of isomers depends on the catalyst used.

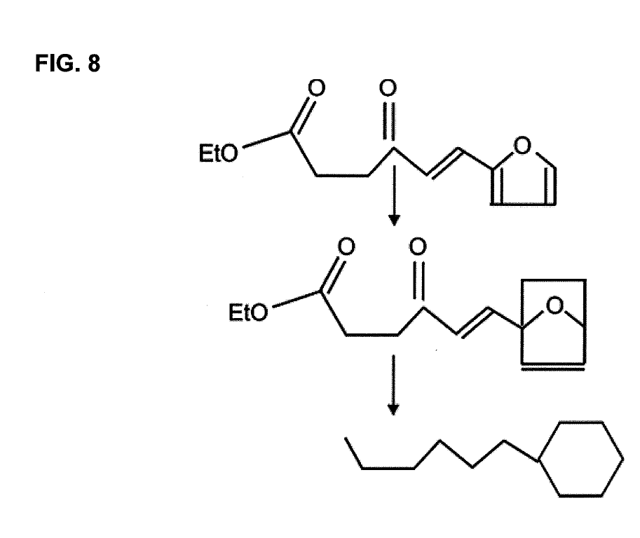

In another embodiment, the furfural by-product or coproduct is also condensed with the levulinic acid or ester to form the furfuryl-substituted levulinates (FIG. 8). Again, depending on the choice of catalyst, β-(or branched) and the δ-(or unbranched) isomers are obtained. Hydroxymethylfurfural also reacts at the aldehyde moiety with levulinates to give a C11 intermediate. Hydroxymethylfurfural is available from renewables by processing sugars with acid catalysts. Fructose has been the preferred sugar substrate for conversion to hydroxymethylfurfural; however, recent reports use CrCl₂ catalyst with glucose as shown in process unit (800).

Three options are available for processing of the furfuryl levulinates. One option is mildhydrogenation to tetrahydrofurans. Another option is to open the furan ring to produce C10 or C11 units. The other option is to conduct a cycloaddition at the furan functionality with a dienophile such as acrolein or acrylic acid (Diels-Alder reaction). The cycloaddition product contains the 7-oxa-bicyclo{2.2.1}heptene moiety with a bridging oxide group that is subsequently removed in the hydrogenation step (400).

The angelica lactone prepared in the third alternative of the first-stage processing (100) is condensed with the aldehyde mixture. The resulting products from this reactant are substituted in the alpha position and can generate isoparaffins in the hydrogenation reactor (400).

Highly reactive ketones will also condense with the levulinate intermediates. These include biacetyl (2,3-butanedione) and 2,3-pentanedione. Both are actually obtained from other reactions of levulinic acid. These highly reactive ketones condense with levulinic acid, resulting in C9 and C10 chains, respectively. Other branched and cyclic ketones are available from pyrolysis of lignin.

Another embodiment utilizes the condensation of levulinate with alpha angelica lactone using a Lewis acid catalyst. The reaction occurs between the enolate of the levulinate and the carbonyl of the enol-activated ester carbonyl group to produce a diketone product.

The condensation of angelica lactone with aldehydes also occurs. The alpha positions are activated by base catalysts, such that condensation with the aldehyde occurs at the alpha position.

Another embodiment utilizes the condensation (Michael reaction) of levulinate with an unsaturated carbonyl compound, such as ethyl acrylate or acrolein, where an alpha carbon of the levulinate reacts with the beta carbon of the unsaturated carbonyl compound. The preferred catalyst is a coordinating metal ion catalyst to promote enolization of the levulinate. Catalysts include zinc, nickel, and other transition metal ions, as well as titania, alumina, and zirconia.

Another embodiment produces cyclic ketones via the Dieckmann condensation of beta-ketoesters and beta-diketone [00047] with the levulinate ester carbonyl group. These cyclic ketones have the advantage that they are easily hydrogenated to cycloparaffins without formation of cyclic ethers.

An alternative condensation method combines an olefinic group with a carbonyl compound. This reactant generates a free radical from reaction of manganese(III) acetate with the carbonyl compound, which subsequently combines with the olefin. With levulinate, this could happen two different ways: 1) reaction of ethyl levulinate radical with an added olefin (FIG. 9A) or 2) reaction of an added ester with the double bond of angelica lactone (FIG. 9B) which is produced in a prior dehydration reaction from either levulinic acid or levininate ester.

### 5. Reagent Aldehyde and Ester Production Unit

Aldehydes are potentially available from a variety of renewable or petrochemical resources. The preferred aldehyde intermediates are those that undergo minimal or no self-condensation. The class comprises aldehydres with no hydrogens on the alpha carbon, such as furfuraldehyde and benzaldehyde, and aldehydes with branching at the alpha carbon, such as isobutyraldehyde and cyclohexanecarboxaldehyde, which inhibits self-condensation.

Reagent aldehydes are formed by dehydration of alcohols over a Cu or Pt catalyst. Precursor alcohols are prepared via Guerbet synthesis or homologation of lower alcohols with carbon monoxide. For example, isobutanol is prepared brom ethanol and methanol using a solid basic Guerbet catalyst. It is also the main product from H₂ and CO at the Leuna Plant. A variety of higher alcohols are present in fusel oil, a by-product from distillation of ethanol from yeast fermentation. Isobutyraldehyde is prepared commercially by oxo reactions of propylene.

Aldehydes are also prepared directly from lower alcohols by Guerbet synthesis at higher temperatures (>400° C.).

Furfural is produced from the thermal decomposition of 5-carbon sugars. Alkoxymethylfurfural is produced from the acid-catalyzed depolymerization of cellulose and starch at lower temperatures.

Cyclohexenylcarboxaldehydes are produced by the cycloaddition of acrolein (from glycerol or lactic acid) with butadiene, from the condensation of ethanol (Lebedev process), or the reaction of acetaldehyde with an olefin (Prins reaction).

C6 and C9 aliphatic aldehydes are formed from oxidation of fatty acids or triglycerides, preferably tall oil fatty acids when integrated with the Kraft process.

Benzaldehydes are available from a variety of renewable sources and by the oxidation of lignin. Lignin may be recovered from solids separated in unit (150) and processed in the reactor (180).

Michael reactions are also conducted with ethyl acrylate, obtained from dehydration of ethyl lactate. Lactic acid from fermentation of the starches is esterified in unit 200. Ethyl lactate is converted catalytically to ethyl acrylate, which condenses at unsaturated carbon (Michael reaction) in the condensation reactor 200.

### 6. Catalytic Hydrogenation Units

A catalytic hydrogenation is performed on the ketoacid and ketoester intermediate produced in the condensation unit (200). These oxygen functional groups are reduced with unsaturation, resulting in formation of the mixtures of paraffins, isoparaffins, cycloparaffins, and alkylaromatics in a hydrogen atmosphere in the hydrogenation reactor (400) (FIGS. 11A and B). Under milder conditions, a tetrahydrofuran ring forms (FIG. 11C). The substituted tetrahydrofurans are utilized as solvents or are blended with hydrocarbon fuels or alcohol-based fuels.

Hydrotreatment of the C6-C8 condensation products using an isomerization catalyst results in branched hydrocarbons suitable for gasoline.

Severe hydrogenation of the C9 to C14 condensation products gives both linear and branched hydrocarbons of appropriate chain lengths for kerosene for the production of jet fuel such as Jet A, Jet A1, JP-5, and JP-8. In addition, cycloparaffins are available from Diels-Alder reactions of the intermediates prepared from ethyl levulinate.

It is advantageous for both fuel properties and processing that the trialkylglycerides or tall oil fatty acids extracted in the oil extraction unit (55) are directly processed by the hydrogenation reactor (400) together with the condensation products. Similarly, turpentine extracted from the Kraft process may undergo an aromatization reaction of its main terpene with reagents such as iodine or PCl3, leading to cymene which then can be hydrotreated to cycloparaffin.

### 7. Chemical Synthesis Units

Extraction Solvents: The use of methyltetrahydrofuran to extract levulinate from the other reaction components was described. Methyltetrahydrofuran and other furan-derived products can also be utilized to extract fermentation products from their aqueous solutions. Thus butanol present in low concentrations in water can be extracted from the aqueous fermentation broth. Recovery of butanol from the extraction solvent is feasible by distilling if the boiling point of the extracting solution is higher than that of the butanol. Thus the preferred embodiments are the cyclic ethers derived from the levulinate condensation reactions.

Plasticizers. Several synthesis steps are incorporated into the integrated parallel processing plant design that utilizes intermediate reagents produced from the noncellulosic feedstocks as well as the levulinate from the cellulosic feedstock. One of the embodiments is the use of a long-chain unsaturated fatty ester, such as oleate, in the condensation units (200) with levulinate to produce a long-chain keto ester. Typically levulinate does not condense with other esters at the ester carbonyl in the acetoacetic type of condensation. Thus the condensation reaction employed is the free radical condensation with the unsaturated portion of an unsaturated or polyunsaturated fatty ester to give a product ester with a very low vapor pressure and comprises an appropriate mixture of flexible alkyl chains and polar groups which allows it to dissolve in and plasticize a polymer material, such as vinyl chloride. The fatty esters are produced in a transesterification unit from extracted vegetable oils or algal oils.

Another embodiment is the acid-catalyzed reaction of levulinate with a diol or polyol to produce a cyclic acetal (1,3-dioxolane or 1,3-dioxane). One useful embodiment uses ethylene glycol, propylene glycol, or a glycerol monoether or glycidyl ether derived from the noncellulosic biomass, and the product is a dioxolane, alkyldioxolane, or an alkoxymethyl-substituted dioxolane. Other polyol reagents are derived from alkoxy sugars. When the alkyl or alkoxy group in the dioxolane product is long, the vapor pressure is low, and good plasticizer properties are obtained.

When the alkyl or alkoxyl group is short (H, methyl, ethyl), the dioxolane product serves as an intermediate for chemical synthesis, such as condensation reactions resulting in 2-substituted acrylates. Alternatively, for the case of dioxlanes derived from diols, the dioxolane ester is reacted with glycerol to form a glyceride that is valuable for polyester and polyurethane synthesis. This requires reaction of the glyceride with a carbonyl compound, such as formaldehyde or acetone, to restore the ketone group of the levulinate glyceride. The reaction is driven by distillation of the small dioxolane, which then is utilized as a diesel or gasoline additive, depending on the size and number of the alkyl groups attached.

Importantly, the reaction or levulinate or levulinic acid with the glycol or glyceryl derivative in the above examples can utilize the crude levulinate mixture obtained directly in the cellulose depolymerization/decomposition as well as the dilute sulfuric acid present in the mixture. The separation of the product from an aqueous phase (by simple decantation) is facilitated by virtue of the hydrophobicity conferred by the long alkoxy group. Further reaction of the decanted levulinate dioxolane with glycerol or with formaldehyde results in the chemical products as described in the previous paragraphs, or alternatively, dilute acid-catalyzed reaction of the decanted levulinate dioxolane product with a small ketone or aldehyde gives the mixture of ethyl levulinate and new dioxolane fuel components.

We claim:

1. A method for converting a source of C6 sugar into a mixture of hydrotreated compounds comprising:
(a) thermocatalytically reacting a source of C6 sugar to produce a solution comprising levulinic acid or levulinic ester;
(b) condensing at least a portion of the levulinic acid or levulinic ester in solution with at least one of C4-C11 aldehydes, C4-C11 ketones, or C4-C11 esters to produce a condensation product; and
(c) hydrotreating at least a portion of the condensation product to provide a mixture of hydrotreated compounds.

2. The method of claim 1, wherein the source of C6 sugar comprises cellulosic materials, starches, or mixtures of cellulosic materials and starches.

3. The method of claim 1, wherein the source of C6 sugar comprises wood, wood pulp, pulping sludge, particleboard, paper, grass, agricultural by-product, or mixture thereof.

4. The method of claim 1, wherein the source of C6 sugar comprises an agricultural by-product comprising straw, stalks, cobs, beets, beet pulp, seed hulls, bagasse, algae, corn starch, potato waste, sugar cane, or fruit waste.

5. The method of claim 1, wherein the source of C6 sugar comprises a by-product, a waste, or a combination of a by product and a waste.

6. The method of claim 1, wherein the thermocatalytic reaction is conducted with acid in water or alcohol.

7. The method of claim 1, further comprising depolymerizing the source of C6 sugar in a thermal unit to provide a soluble carbohydrate intermediate prior to thermocatalytically reacting to produce the levulinate acid or levulinate ester.

8. The method of claim 7, wherein the soluble carbohydrate intermediate comprises anhydrosugar.

9. The method of claim 8, wherein the thermocatalytic reaction of the anhydrosugar is conducted with a solid acid catalyst.

10. The method of claim 1, wherein the C4-C11 aldehyde is branched or aromatic.

11. The method of claim 10, wherein the C4-C11 aldehyde is selected from the group consisting of isobutyraldehyde, furfural, hydroxymethylfurfural, substituted benzaldehydes, and cyclic aliphatic aldehydes.

12. The method of claim 11, wherein the isobutyraldehyde is prepared by dehydrogenation of isobutyl alcohol.

13. The method of claims 11, wherein the isobutyraldehyde is prepared by condensation of methyl and ethyl alcohols, aldehydes, or mixture thereof.

14. The method of claim 10, wherein the aldehyde is derived via an oxo reaction of an olefin.

15. The method of claim 10, wherein the C4-C11 aldehyde is a cyclic aliphatic aldehyde produced by Diels-Alder reactions of acrolein with butadiene.

16. The method of claim 1, wherein the C4-C11 ketone is selected from the group consisting of 1,2 diketones, 1,2 ketoesters, 2,3-butanedione, and 2,3-pentanedione.

17. The method of claim 1, wherein the C4-C11 ester comprises vinyl ester.

18. The method of claim 1, wherein the C4-C11 ester comprises angelica lactone.

19. The method of claim 1, wherein the condensing comprises condensing in the presence of catalyst.

20. The method of claim 19, wherein the catalyst comprises a solid base catalyst.

21. The method of claim 19, wherein the catalyst comprises hydrotalcite and impregnated hydrotalcite.

22. The method of claim 19, wherein the catalyst for the condensation comprises a free radical initiator.

23. The method of claim 22, wherein the free radial initiator comprises manganese(III) acetate.

24. The method of claim 19, wherein the catalyst for the condensation is a transition metal ion or heterogeneous catalyst comprising titania, zirconia, or alumina.

25. The method of claim 1, further comprising separating the condensation products based on carbon ranges appropriate for jet fuel and diesel.

26. The method of claim 1, wherein the hydrotreating of the condensation product comprises coprocessing with free fatty acids, natural oils, or combinations thereof to diesel or jet fuel blendstocks.

27. The method of claim 1, wherein the hydrotreating comprises hydrotreating a cyclic condensation product to jet fuel blendstock components.

28. The method of claim 26, wherein the condensation product is separated to chain length C10-C15, comprising n-alkanes, isoalkanes, cycloalkanes, and arylalkanes.

29. The method of claim 1, wherein methyltetrahydrofuran and other low carbon by-products are separated for use as amphiphilic solvents.

30. The method of claim 1, wherein the hydrotreating of levulinate condensation products yields cyclic ethers.

31. The method of claim 30, wherein cyclic ethers comprise alkyl tetrahydrofurans.

32. The method of claim 30, wherein a portion of the levulinate condensation product is condensed with a reagent containing an unsaturated group to produce a long-chain keto ester.

33. The method of claim 1, further comprising converting at least a portion of the condensation product to a ketal in an acid-catalyzed reaction with a diol.
